(19) United States
(12) Patent Application Publication
(10) Pub. No.: US 2025/0195052 A1
(43) Pub. Date: Jun. 19, 2025

(54) ENDOCISTERNAL KIT AND METHOD FOR CREATION OF A CEREBRAL THIRD FLOOR VENTRICULOSTOMY USING MINIMALLY INVASIVE TECHNIQUES

(71) Applicant: Deinde Medical Corp., Miramar, FL (US)

(72) Inventors: Robert C. Farnan, Fort Lauderdale, FL (US); Ajay Wakhloo, Weston, MA (US)

(21) Appl. No.: 19/062,311

(22) Filed: Feb. 25, 2025

Related U.S. Application Data
(63) Continuation of application No. PCT/US2023/031014, filed on Aug. 24, 2023.
(60) Provisional application No. 63/401,230, filed on Aug. 26, 2022.

(51) Int. Cl. A61B 17/00; A61B 17/34; A61B 90/00; A61N 5/06

(52) U.S. Cl. CPC A61B 17/00234; A61B 17/3401; A61B 90/37; A61N 5/0601; A61N 5/0622; A61B 2017/00238; A61B 2017/003; A61B 2090/3762; A61N 2005/0612

(57) ABSTRACT

A method of performing a ventriculostomy includes creating an access site in a lumbar region. A guidewire is inserted into the access site. A sheath is introduced over the guidewire, into the access site, and the tip of the sheath is positioned in a subarachnoid space. A first catheter and a second catheter are introduced through the access site. The first catheter and the second catheter are guided proximate to a ventricle membrane of a ventricle. A hole is created in the ventricle membrane and at least one of the first catheter or the second catheter are advanced through the hole. The hole in the ventricle membrane is dilated. Contrast is injected into the ventricle to confirm cerebrospinal fluid is draining into a subarachnoid cistern.

### CROSS REFERENCE TO RELATED APPLICATIONS

This application claims priority to U.S. Provisional Patent Application No. 63/401,230 filed Aug. 26, 2022 (expired) and PCT Patent Application No. PCT/US2023/031014 filed Aug. 24, 2023 (pending), the disclosures of which are incorporated herein by reference in their entirety.

This application is also generally related to the subject matter disclosed in U.S. Provisional Patent Application No. 63/041,395 filed Jun. 19, 2020 (expired), PCT Patent Application No. PCT/US2021/037803 filed Jun. 17, 2021 (expired), and U.S. patent application Ser. No. 18/080,995, filed Dec. 14, 2022 (pending), the disclosures of which are incorporated herein by reference in their entirety.

### TECHNICAL FIELD

The present disclosure generally relates to minimally invasive surgical procedures and devices and, more particularly, to minimally invasive neurosurgical procedures for treating non-communicating hydrocephalus. The disclosure also relates to minimally invasive neurosurgical procedures for Photobiomodulation Therapy.

### BACKGROUND

The present disclosure contemplates that treatments for some conditions may include surgically creating openings through tissues (e.g., biological tissue membranes). For example, for patients suffering from non-communicating hydrocephalus, the normal channel for drainage of the cerebrospinal fluid (“CSF”) from the third ventricle to the lower CSF chambers and central canal that leads to the spinal cord is blocked by an occlusion within the cerebral aqueduct. These patients, through the normal production of the CSF, experience a “rise” in the pressure of the lamina and third ventricles, causing these fluid chambers to distend. To relieve the pressure, neurosurgeons have used a procedure called a third ventriculostomy to rupture the membrane situated on the floor of the third ventricle. This procedure requires creating a hole through the patient's skull and moving brain tissue to allow the introduction and navigation of an endoscope to the desired site for creation of the ventriculostomy. Once the membrane is visible in the endoscopic image, a balloon catheter (e.g., Fogarty) is advanced up to and through the membrane. Once the balloon portion of the catheter is passed through the membrane, it is inflated and then retracted through the membrane to create the fenestration (e.g., hole) between the CSF chambers. An alternative ventriculostomy procedure uses similar access through the skull and brain, but, instead of the endoscope/balloon catheter combination, a surgical instrument is used that discretely cauterizes the membrane as it is opened. While such procedures have been successfully used on patients, these procedures involve risks and disadvantages associated with surgically penetrating the skull, dura matter, and/or brain tissues.

The present disclosure further contemplates that treatments for some conditions may include Photobiomodulation (PBM Therapy) which is the application of red and near infra-red light for medical treatment. The light can originate from a laser or a light emitting diode (LED), and can be pulsed and/or continuous. The health benefits of PBM Therapy are well documented with the application of light typically to the exterior of the body of the patient in a non-invasive manor. Generally, transcranial PBM procedures involve the placement of a light source, or multiple light sources, on one or more areas of the patient's head, with the goal of treating a certain part of the brain. In transcranial PBM, light must pass through layers of tissue including the scalp, periosteum, skull bone, meninges, and dura, before reaching the cortical surface of the brain. Due to the exponential attenuation of light during the journey through the skull and brain tissues, only a small fraction of the incident light may be delivered to the intended tissue. Devices and methods of delivering PBM therapy more directly to the tissue to be treated, and specifically the brain and/or spinal tissue, may provide unique benefits.

Accordingly, and despite the various advances already made in this field, there is a need for further improvements related to minimally invasive neurosurgical procedures and related devices.

### SUMMARY

Generally, a method of performing a ventriculostomy is provided, the method including creating an access site in the tissue enveloping the spinal cord in the lumbar region, inserting a guidewire into the access site, introducing a sheath over the guidewire, introducing a dilator over the guidewire, and dilating the access site. The sheath is inserted into the access site, the tip of the sheath is positioned in a subarachnoid space of the spinal column, and the dilator is removed. A first catheter is introduced into the sheath and through the access site. A second catheter is introduced into the sheath and through the access site. The first catheter and the second catheter are guided proximate to a ventricle membrane of a ventricle. The ventricle membrane is engaged with the tip of the first catheter and a hole is created in the ventricle membrane. At least one of the first catheter or the second catheter is advanced through the hole in the ventricle membrane. An expandable element is advanced through the hole created in the ventricle membrane and the expandable element is centered in the hole. The expandable element is expanded to dilate the hole in the ventricle membrane and the expandable element is contracted. Contrast is injected into the ventricle to confirm cerebrospinal fluid is draining into a subarachnoid cistern. The expandable element, the second catheter, the first catheter, the guidewire and the sheath are removed. A blood patch is applied to the access site.

In some embodiments, creating the access site in the tissue enveloping the spinal cord includes piercing the tissue enveloping the spinal cord with an 18 to 21 gauge needle. The needle may include a stylet. The method may include using an introducer to create the access site in the tissue enveloping the spinal cord. The diameter of the guidewire may be 0.014″ to 0.025″. The guidewire may be 70 to 100 cm long. The dilator may be 4F to 7F dilator. Dilating the access site may include staged dilation of the access site. The dilator may be a plurality of dilators. Dilating the access site may include dilating the access site with a 4F dilator, dilating the access site with a 5F dilator, dilating the access site with a 6F dilator, and dilating the access site with a 7F dilator. The sheath may be 40 to 60 cm long. The ventricle may be a third ventricle and the ventricle membrane may be a floor of the third ventricle. The second catheter may be a neurovascular distal access catheter. The first catheter may be a steerable catheter. The first catheter may be a microcatheter. The first catheter may be longer than the second catheter. The second catheter may be 120 to 130 cm long. The first catheter may be 150 to 160 cm long.

In alternative or additional aspects, guiding the first catheter and the second catheter proximate to the ventricle membrane may include guiding the first catheter and the second catheter along an anterior side of the spinal cord. Guiding the first catheter and the second catheter proximate to the ventricle membrane may include guiding the first catheter and the second catheter into a central canal of the brain. Guiding the first catheter and the second catheter proximate to the ventricle membrane may include guiding the first catheter and the second catheter with a tip of at least one of the first catheter and the second catheter pointing upward towards a clivus and away from a basilar artery. Guiding the first catheter and the second catheter proximate to the ventricle membrane may include guiding the first catheter and the second catheter to a mid-line of the brain and avoiding lateral recesses on either side of the mid-line. Guiding the first catheter and the second catheter proximate to the ventricle membrane may include visualizing at least one of the first catheter and the second catheter using an imaging device. Guiding the first catheter and the second catheter proximate to the ventricle membrane may include visualizing the ventricle membrane using an imaging modality. The imaging modality may include computed tomography. The imaging modality may include fluoroscopic computed tomography. Visualizing at least one of the first catheter and the second catheter may include co-registering with magnetic resonance imaging.

In alternative embodiments, guiding the first catheter and the second catheter proximate to the ventricle membrane may include rotating the first catheter. Guiding the first catheter and the second catheter proximate to the ventricle membrane may include positioning the tip of the first catheter between two mamillary bodies. Guiding the first catheter and the second catheter proximate to the ventricle membrane may include positioning the tip of the first catheter to engage the ventricle membrane. Removing the first catheter may include leaving a tip of the second catheter in the ventricle. Removing the first catheter may include leaving the tip of the second catheter in the ventricle. The second catheter may provide a protective conduit to the brain along the spinal cord and brain structure, minimizing potential damage to brain structures and nerves.

In alternative or additional aspects, the expandable element may be a balloon catheter. The method may include inflating the balloon catheter. The method may include deflating the balloon catheter. The expandable element may be configured to expand to at least 5 mm in diameter. The hole in the ventricle membrane may be created with the guidewire. The first catheter may be introduced into the second catheter before the first catheter and the second catheter are introduced into the sheath. The expandable element may be introduced through the second catheter before advancing the expandable element through the hole created in the ventricle membrane. The first catheter may be removed before introducing the expandable element through the second catheter. The second catheter may be advanced to capture the expandable element. The second catheter may be retracted from the hole created in the ventricle membrane before centering the expandable element in the hole created in the ventricle membrane.

In some embodiments, the guidewire is a first guidewire, and the method includes introducing a second guidewire into the sheath and through the access site, guiding the second guidewire proximate to the ventricle membrane, advancing the second guidewire through the hole created in the ventricle membrane, and removing the second guidewire. The second guidewire may be introduced into the first catheter before the second guidewire and the first catheter are introduced into the sheath. The second guidewire may be introduced into the first catheter and the first catheter may be introduced into the second catheter before the second guidewire, the first catheter, and the second catheter are introduced into the sheath. The hole in the ventricle membrane may be created with the second guidewire. The diameter of the second guidewire may be 0.014″. The second guidewire may be an extra support guidewire. The method may include advancing the second guidewire to the tip of the first catheter.

In alternative or additional aspects, guiding the second guidewire, the first catheter, and the second catheter proximate to the ventricle membrane may include leading with the second guidewire. Guiding the second guidewire, the first catheter, and the second catheter proximate to the ventricle membrane may include pulling a tip of the second guidewire back from the tip of the first catheter. Pulling the tip of the second guidewire back from the tip of the first catheter may improve steering of the first catheter. Removing the first catheter may include leaving a tip of the second guidewire in the ventricle. Guiding the second guidewire, the first catheter, and the second catheter proximate to the ventricle membrane may include guiding the second guidewire, the first catheter, and the second catheter along an anterior side of the spinal cord. Guiding the second guidewire, the first catheter, and the second catheter proximate to the ventricle membrane may include guiding the second guidewire, the first catheter, and the second catheter into a central canal of the brain. Guiding the second guidewire, the first catheter, and the second catheter proximate to the ventricle membrane may include guiding the second guidewire, the first catheter, and the second catheter with a tip of at least one of the second guidewire, the first catheter, and the second catheter pointing upward towards a clivus and away from a basilar artery. Guiding the second guidewire, the first catheter, and the second catheter proximate to the ventricle membrane may include guiding the second guidewire, the first catheter, and the second catheter to a mid-line of the brain and avoiding lateral recesses on either side of the mid-line.

In alternative embodiments, guiding the second guidewire, the first catheter, and the second catheter proximate to the ventricle membrane may include visualizing at least one of the second guidewire, the first catheter, and the second catheter using an imaging device. Guiding the second guidewire, the first catheter, and the second catheter proximate to the ventricle membrane may include visualizing the ventricle membrane using an imaging modality. The imaging modality may include computed tomography. The imaging modality may include fluoroscopic computed tomography. Visualizing at least one of the second guidewire, the first catheter, and the second catheter may include co-registering with magnetic resonance imaging.

Generally, a system for performing a ventriculostomy is provided. The system includes a needle, a guidewire, a sheath, a dilator, a first and second catheter, and an expandable element. The needle is configured for performing a lumbar stick. The sheath is configured for insertion into a subarachnoid space of a spinal column anterior to a spinal cord. The dilator is configured for use with the sheath. The second catheter is configured for use with the first catheter. The expandable element is configured to dilate a membrane.

In some embodiments, the needle may be an 18 to 21 gauge needle. The needle may include a stylet. The needle may include an introducer. The diameter of the guidewire may be 0.014″ to 0.025″. The guidewire may be 70 to 100 cm long. The dilator may be a 4F to 7F dilator. The dilator may be a plurality of dilators including a 4F dilator, a 5F dilator, a 6F dilator, and a 7F dilator. The dilator may be a staged dilator. The sheath may be 40 to 60 cm long. The second catheter may be a neurovascular distal access catheter. The first catheter may be a steerable catheter. The first catheter may be a microcatheter. The first catheter may be longer than the second catheter. The second catheter may be 120 to 130 cm long. The first catheter may be 150 to 160 cm long. The expandable element may be a balloon catheter. The expandable element may be expandable to at least 5 mm in diameter.

An alternative system for performing a ventriculostomy is provided. The alternative system includes a needle, a first and second guidewire, a sheath, a dilator, a first and second catheter, and an expandable element. The needle is configured for performing a lumbar stick. The sheath is configured for insertion into a subarachnoid space of a spinal column anterior to a spinal cord. The dilator is configured for use with the sheath. The second catheter is configured for use with the first catheter. The expandable element is configured to dilate a membrane.

In some embodiments, the needle may be an 18 to 21 gauge needle. The needle may include a stylet. The needle may include an introducer. The diameter of the first guidewire may be 0.014″ to 0.025″. The first guidewire may be 70 to 100 cm long. The dilator may be a 4F to 7F dilator. The dilator may be a plurality of dilators including a 4F dilator, a 5F dilator, a 6F dilator, and a 7F dilator. The dilator may be a staged dilator. The sheath may be 40 to 60 cm long. The diameter of the second guidewire may be 0.014″. The second guidewire may be an extra support guidewire. The second catheter may be a neurovascular distal access catheter. The first catheter may be a steerable catheter. The first catheter may be a microcatheter. The first catheter may be longer than the second catheter. The second catheter may be 120 to 130 cm long. The first catheter may be 150 to 160 cm long. The expandable element may be a balloon catheter. The expandable element may be expandable to at least 5 mm in diameter.

Another system for performing a ventriculostomy is provided. The system includes an 18 to 21 gauge needle, a first guidewire, a second guidewire, a sheath, a dilator, a first catheter, a second catheter, and an expandable element. The needle is configured for performing a lumbar stick. The first guidewire has a diameter of 0.014″ to 0.025″ and a length of 70 to 100 cm. The second guidewire has a diameter of 0.014″. The sheath is configured for insertion into a subarachnoid space of a spinal column anterior to a spinal cord and is 40 to 60 cm long. The dilator is configured for use with the sheath. The first catheter is a steerable microcatheter. The second catheter is a neurovascular distal access catheter configured to fit over the first catheter. The expandable element is configured to dilate a membrane and is expandable to at least 5 mm diameter. The first catheter is longer than the second catheter.

In alternative or additional aspects, the needle includes a stylet. The needle may include an introducer. The dilator may be a 4F to 7F dilator. The dilator may be a plurality of dilators including a 4F dilator, a 5F dilator, a 6F dilator, and a 7F dilator. The dilator may be a staged dilator. The second guidewire may be an extra support guidewire. The second catheter may be 120 to 130 cm long. The first catheter may be 150 to 160 cm long. The expandable element may be a balloon catheter.

A method of guiding a first catheter to guide a second catheter is provided, the method including configuring the first catheter inside the second catheter, introducing the first catheter and the second catheter into a body of a patient, and guiding the first catheter thereby guiding the second catheter.

In some embodiments, guiding the second catheter with the first catheter includes rotating the first catheter. The method may include removing the first catheter from the second catheter. The second catheter may provide a protective conduit for other devices to be inserted into and/or through the second catheter. The method may include introducing the first catheter and the second catheter through a sheath. The second catheter may be a neurovascular distal access catheter. The first catheter may be a steerable catheter. The first catheter may be a microcatheter. The first catheter may be longer than the second catheter. The method may include introducing the first catheter and the second catheter over a guidewire. The method may include guiding the guidewire, the first catheter, and the second catheter with the first catheter. The method may include advancing the second guidewire to the tip of the first catheter. The method may include guiding the first catheter and/or the second catheter with the guidewire.

In alternative embodiments, the method may include pulling the tip of the guidewire back from the tip of the first catheter. Pulling the tip of the guidewire back from the tip of the first catheter may improve the steering of the first catheter. The method may include removing the first catheter from the second catheter without removing the guidewire from the second catheter. The method may include removing the guidewire from the first catheter and/or the second catheter. The method may include guiding the guidewire, the first catheter, and the second catheter proximate to a membrane. The method may include engaging the membrane with the tip of the first catheter. The method may include creating a hole in the membrane and advancing the guidewire and first catheter through the hole created in the membrane. The method may include creating a hole in the membrane with the guidewire. The method may include advancing the second catheter through the hole created in the membrane. The method may include removing the first catheter from the second catheter. The method may include introducing an expandable element through the second catheter and the hole created in the membrane. The method may include retracting the second catheter from the hole created in the membrane.

In alternative or additional aspects, the method may include centering the expandable element in the hole created in the membrane. The method may include expanding the expandable element to dilate the hole created in the membrane. Expanding the expandable element may include inflating a balloon catheter. The method may include contracting the expandable element. Contracting the expandable element may include deflating a balloon catheter. The expandable element may be configured to expand to at least 5 mm diameter. The method may include advancing the second catheter to capture the expandable element. The expandable element may be a balloon catheter. The method may include removing the expandable element.

In some embodiments, the method may include removing the second catheter. The method may include removing the guidewire. The guidewire may be an extra support guidewire. Guiding the first catheter and/or the second catheter may include visualizing at least one of the first catheter and the second catheter using an imaging modality. The imaging modality may include computed tomography. The imaging modality may include fluoroscopic computed tomography. Visualizing at least one of the first catheter and the second catheter may include co-registering with magnetic resonance imaging.

In some embodiments, the method may include applying light to the brain and/or spinal cord tissue. The method may include advancing at least one of the first catheter and the second catheter proximate to brain and/or spinal cord tissue, and applying light to the brain and/or spinal cord tissue. The method may include energizing a light element. Energizing the light element may include supplying light to the light element.

A system for guiding a first catheter to guide a second catheter is provided. The system includes a first catheter and a second catheter configured to fit over the first catheter. In some embodiments, the first catheter may be a steerable catheter. The first catheter may be a microcatheter. The second catheter may be a neurovascular distal access catheter. The first catheter may be longer than the second catheter. The system may include a guidewire. The guidewire diameter may be 0.014″ to 0.025″. The guidewire may be an extra support guidewire. The system may include a sheath configured to fit over the second catheter. The system may include an expandable element configured to dilate a membrane. The expandable element may be a balloon catheter.

The devices, systems, and/or methods disclosed herein may include any combination of apparatus, elements, and/or methods disclosed herein. Additional features and advantages of the inventive aspects will become more apparent upon review of the following detailed description taken together with accompanying drawings of the illustrative and exemplary embodiments.

### BRIEF DESCRIPTION OF DRAWINGS

FIG. 1 is an illustrative section view of a lumbar puncture location on a patient.

FIG. 2 is an illustrative section view of a lumbar puncture being performed on a patient.

FIG. 3 is a section view of an illustrative first guidewire being inserted through an access site created by the lumbar puncture of FIG. 2.

FIG. 4 is an isometric view of an illustrative sheath and dilator.

FIG. 5 is an illustrative section view of the sheath and dilator of FIG. 4 being guided onto the first guidewire of FIG. 3.

FIG. 6 is an illustrative section view of the dilator of FIG. 4 being used to dilate the access site of FIG. 3.

FIG. 7 is another illustrative section view of the dilator of FIG. 4 being used to dilate the access site of FIG. 3.

FIG. 8 is an illustrative isometric view of the sheath of FIG. 4 in a subarachnoid space anterior to a spinal cord of the patient.

FIG. 9 is an illustrative view of a second guidewire being introduced into a first catheter, and the first catheter being introduced into a second catheter.

FIG. 9A is an illustrative view of an assembly of the second guidewire, the first catheter, and the second catheter of FIG. 9.

FIG. 10 is an illustrative view of the second guidewire, the first catheter, and the second catheter of FIG. 9A being directed through the sheath of FIG. 4.

FIG. 10A is an enlarged view of the tips of the second guidewire, the first catheter, and the second catheter of FIG. 10.

FIG. 11 is an illustrative view of the second guidewire, the first catheter, and the second catheter of FIG. 9A being directed along an anterior side of a spinal cord.

FIG. 11A is an illustrative view of the second guidewire, the first catheter, and the second catheter of FIG. 9A being directed along the midline of the brain.

FIG. 12 is an illustrative view of the second guidewire, the first catheter, and the second catheter of FIG. 9A being directed within a brain of the patient.

FIG. 13 is an illustrative view of the first catheter of FIG. 9 “tenting” the membrane floor of the third ventricle.

FIG. 14 is an illustrative view showing advancing the second guidewire and the first catheter of FIG. 9 through the membrane of FIG. 13.

FIG. 15 is an illustrative view showing the second catheter of FIG. 9 traversing the membrane floor of the third ventricle, the second guide wire in the third ventricle, and the first catheter removed from the second catheter.

FIG. 16 is an illustrative view of an expandable element being centered in an opening in the membrane of FIG. 13.

FIG. 17 is an illustrative view showing expanding the expandable element of FIG. 16 to dilate the opening in the membrane of FIG. 13.

### DETAILED DESCRIPTION

Illustrative embodiments according to at least some aspects of the present disclosure are described and illustrated below and include devices and methods relating to surgical procedures. Of course, it will be apparent to those of ordinary skill in the art that the embodiments discussed below are examples and may be reconfigured without departing from the scope and spirit of the present disclosure. It is also to be understood that variations of the exemplary embodiments contemplated by one of ordinary skill in the art shall concurrently comprise part of the instant disclosure. However, for clarity and precision, the illustrative embodiments as discussed below may include optional steps, methods, and features that one of ordinary skill should recognize as not being a requisite to fall within the scope of the present disclosure.

The present disclosure includes, among other things, catheters and steerable catheters. As used herein, the terms catheter and steerable catheter may include catheters with a dilation mechanism and/or other additional features and functions. Some illustrative embodiments according to at least some aspects of the present disclosure may be used in connection with guiding medical devices and/or creating openings in tissues during minimally invasive neurosurgical procedures.

Illustrative apparatus and methods disclosed herein may be utilized in connection with minimally invasive surgical treatment of hydrocephalus involving creation of an opening through the membrane floor of the third ventricle. While the present detailed description of illustrative embodiments focuses on minimally invasive neurosurgical procedures, it will be appreciated that various embodiments according to at least some aspects of the present disclosure may be utilized in connection with other procedures. Additionally, although some features may be described herein in connection with particular exemplary embodiments, one of ordinary skill in the art will appreciate that any features described herein may be used alone or in any combination within and between various embodiments.

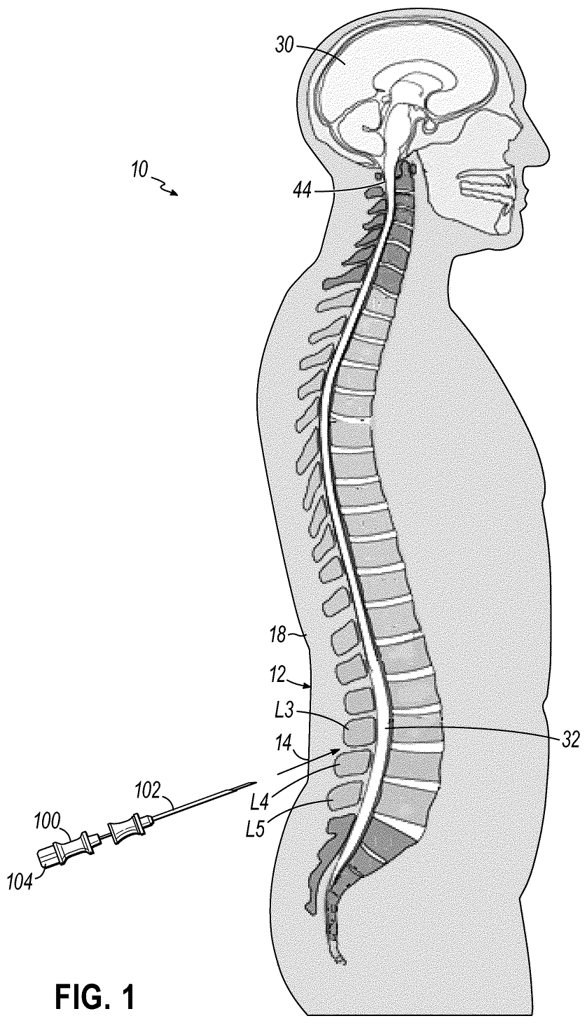

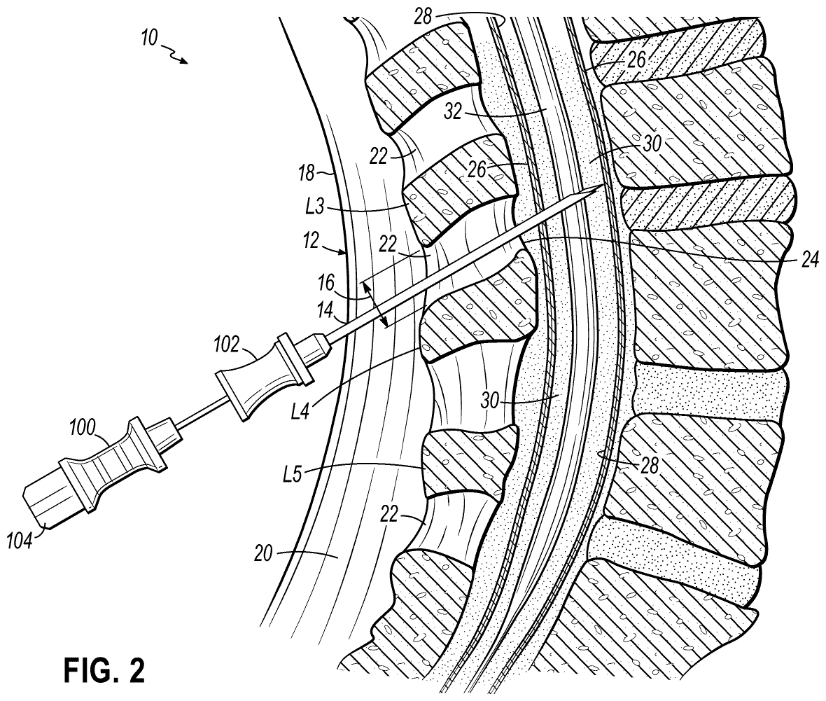

FIG. 1 is an illustrative section view of a lumbar puncture (spinal tap) location on a patient, and FIG. 2 is an illustrative section view of a lumbar puncture being performed on a patient, all according to at least some aspects of the present disclosure. In this illustrative method for performing a ventriculostomy, a lumbar puncture is performed by a surgeon with a needle 100 and introducer 102 to create an access site 14 in a lumbar region 12 of a patient 10. Generally, a lumbar puncture is performed between the L3 and L4 vertebra or the L4 and L5 vertebra of a patient 10. The lumbar puncture may be performed using the same devices that are commonly used for sampling the CSF from the subarachnoid space 30 of a patient 10 and generally include a 18-21 gauge hollow needle with a stylet. In this example, the needle 100 is a 21 gauge hollow needle and includes a stylet 104. During the lumbar puncture, the patient 10 may lie on an operating table, for example, on their side with their chin tucked to their chest and their knees tucked to their abdomen so that their back is arched (the fetal position) which helps to widen a distance 16 between the vertebrae. The needle 100 is inserted through the patient's skin 18, subcutaneous tissue 20, interspinous ligament 22, ligamentum flavum 24, dura matter 26, arachnoid matter 28, and into the subarachnoid space 30 where the CSF is located. Once the needle 100 has penetrated the subarachnoid space 30, CSF may begin to drip out of the needle 100 indicating to the surgeon that the needle 100 has reached the subarachnoid space 30. Alternate methods of accessing the subarachnoid space 30 may include a C1-C2 puncture into the CSF system, for example.

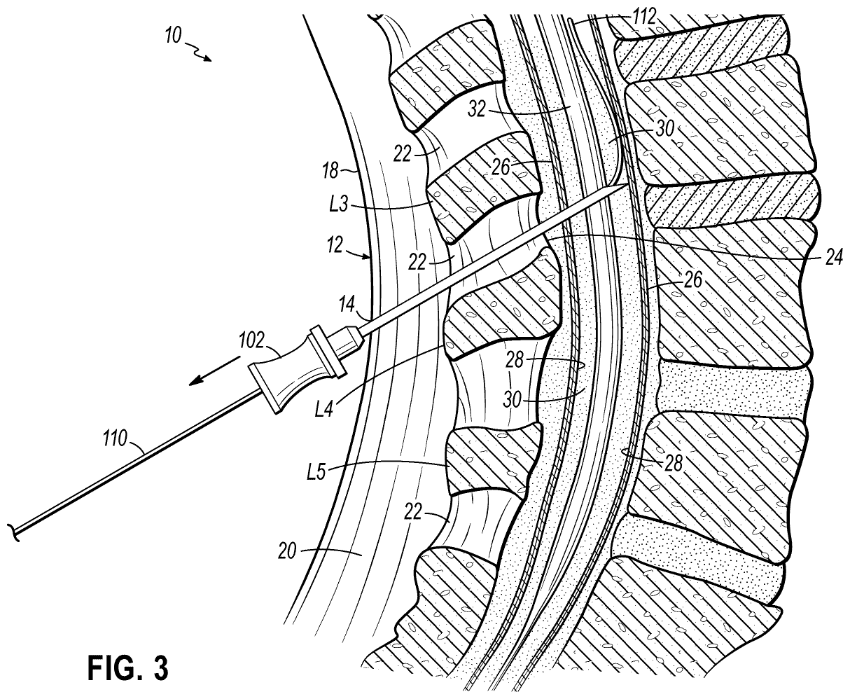

FIG. 3 is a section view of an illustrative first guidewire 110 being inserted through the access site 14 created by the lumbar puncture illustrated in FIG. 2, according to at least some aspects of the present disclosure. In this illustrative method, a tip 112 at the distal end of the first guidewire 110 is inserted through the introducer 102 and the access site 14 and between the L3 and L4 vertebra. In this illustrative embodiment, the first guidewire 110 is 0.014″-0.025″ diameter and 70-100 cm long. After the first guidewire 110 is inserted through the introducer 102, the introducer 102 is withdrawn proximally from the access site 14 and is removed from a proximal end of the first guidewire 110.

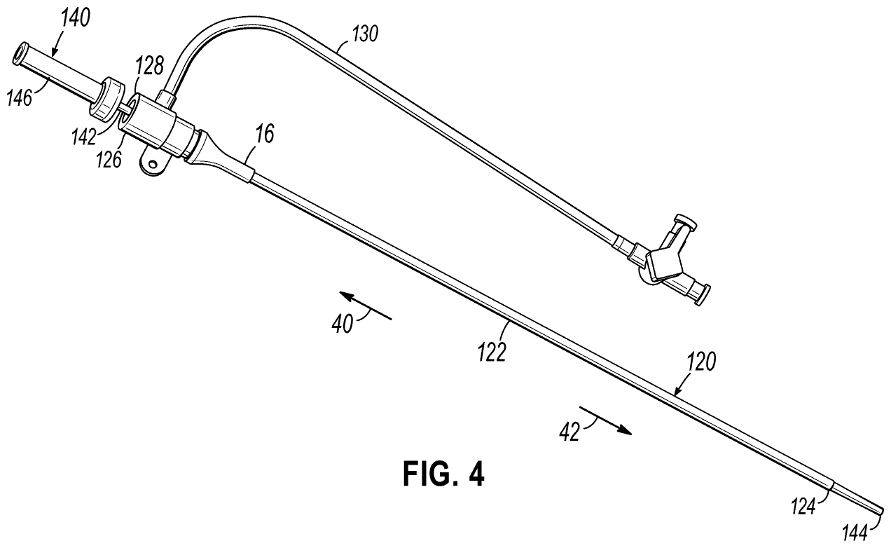

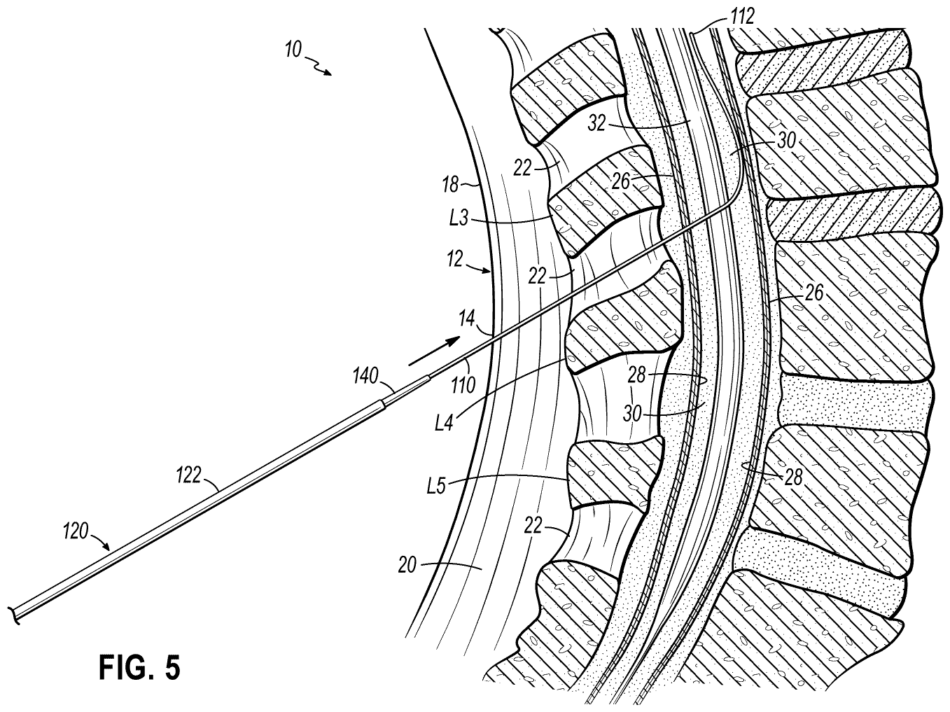

FIG. 4 is an illustrative isometric view of a sheath 120 with a dilator 140, and FIG. 5 is an isometric view of the sheath 120 and dilator 140 being guided onto the first guidewire 110, all according to at least some aspects of the present disclosure. The illustrative sheath 120 includes an elongated shaft 122, a tip 124 located at a distal end of the shaft 122, a proximal end 126, and a sheath lumen extending longitudinally through the sheath 120 from the proximal end 126 to the tip 124. The sheath lumen is generally located along a longitudinal centerline of the shaft 122 and is configured to slidably receive medical devices such as guidewires, catheters, and/or dilators, for example. In this illustrative example, the sheath 120 is 40-60 cm long. In some exemplary embodiments, at least a portion of the sheath 120 may be constructed of a radiopaque material which may facilitate visualization of the sheath 120 using various medical imaging modalities.

The sheath 120 includes an internal seal at the proximal end 126 of the sheath lumen. The internal seal allows a user to access the proximal end 126 of the sheath lumen with guidewires, catheters, and/or dilators, for example, without significant loss of bodily fluids. The internal seal remains in a closed position until a device, such as a dilator for example, is inserted into the sheath lumen, and then the internal seal will seal around the device. The proximal end 126 of the sheath 120 includes a lock 128 which may be used with a device, such as a dilator for example, to secure the device into the sheath 120 prior to a procedure, such as tissue dilation for example. The lock 128 allows the device to be unsecured and removed from the sheath 120 when the procedure is complete. The proximal end 126 of the sheath 120 includes a side port 130 which may be used to flush the sheath lumen of air at the start of a procedure and/or inject contrast, pharmaceuticals, or other fluids, for example.

As used herein to describe various embodiments from the perspective of a user of a surgical device, “proximal” may refer to a direction generally towards the user of the device, while “distal” may refer to a direction generally away from the user of the device. Similarly, in the context of a surgical device inserted into a patient's body and from the perspective of a user of the device, “proximal” may refer to a direction generally away from the center of the patient's body, and “distal” may refer to a direction generally towards the center of the patient's body. For reference, arrow 40 points generally proximally and arrow 42 points generally distally.

The illustrative dilator 140 includes an elongated shaft 142, a tip portion 144 located at a distal end of the shaft 142, a proximal end 146, and a lumen extending longitudinally through the dilator 140 from the proximal end 146 to the tip 144. The dilator lumen is generally located along a longitudinal centerline of the shaft 142 and is configured to slidably receive medical devices, such as guidewires for example. The tip 144 is configured to allow the dilator 140 to be advanced through biological tissue to dilate an opening in the tissue, for example. In some exemplary embodiments, at least a portion of the dilator 140 may be constructed of a radiopaque material which may facilitate visualization of the dilator 140 using various medical imaging modalities.

In this illustrative method, the tip 144 of the dilator 140 is inserted into the sheath lumen at the proximal end 126 of the sheath 120 and the dilator 140 is advanced through the sheath 120 until the tip 144 of the dilator 140 extends past the tip 124 of the sheath 120. The sheath 120 and the dilator 140 are introduced over the first guidewire 110 by feeding the proximal end of the first guidewire 110 into the lumen at the tip 144 of the dilator 140. The sheath 120 and the dilator 140 are advanced along the first guidewire 110 until the tip 144 of the dilator 140 is near the access site 14. In alternate embodiments the first guidewire 110, sheath 120, and dilator 140 may be assembled prior to the first guidewire 110 being inserted through the access site 14.

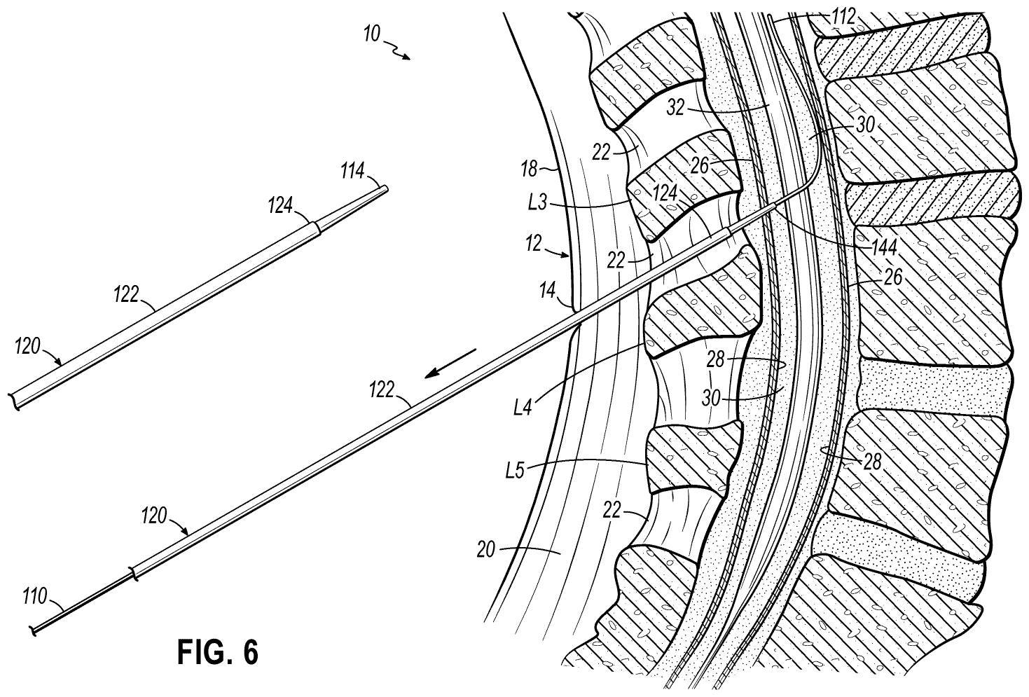

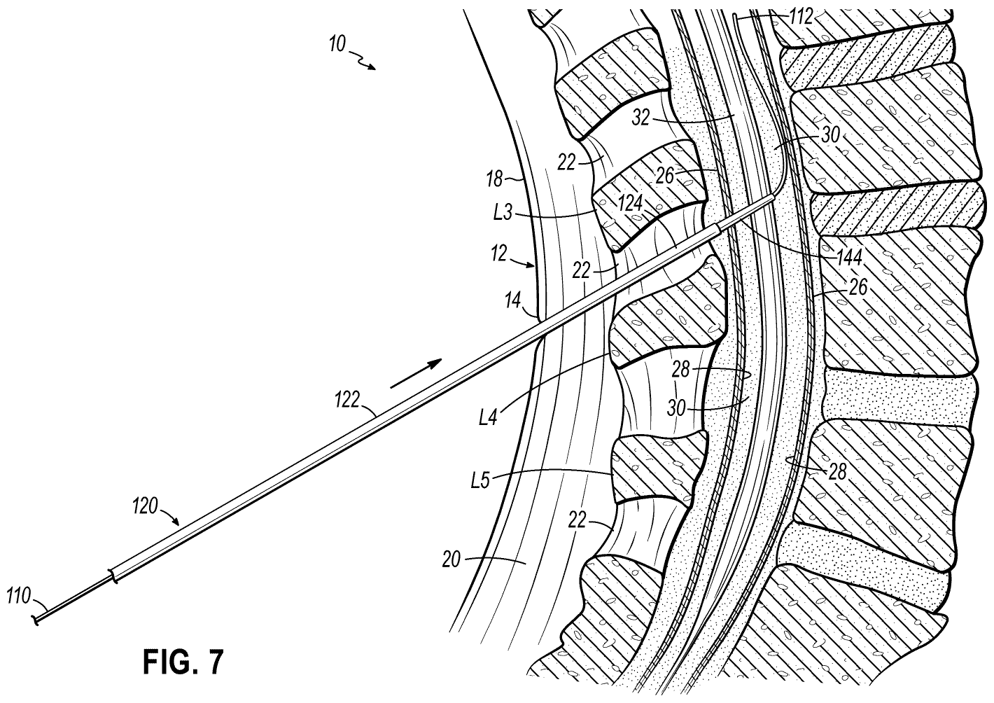

FIGS. 6 and 7 are illustrative section views of the dilator 140 being used to dilate the access site 14, according to at least some aspects of the present disclosure. In this illustrative method, the access site 14 is dilated to allow the sheath 120 to be inserted into the access site 14, between the L3 and L4 vertebra, and into the subarachnoid space 30. In this illustrative method, the access site 14 is dilated using staged dilation of the dura matter 26 to gradually obtain enough dilation to then introduce the sheath 120. Preparing the access site 14 for insertion of the sheath may include a combination of dilation of tissue and guiding the sheath 120 and/or dilator 140 around tissue. The dilator 140 may be a staged dilator, a series of dilators, or any other device configured to dilate a tissue opening. In this illustrative example, the dilator 140 is a series of dilators ranging in size from 4F to 7F. The surgeon begins dilating the access site 14 by advancing the tip 144 of a 4F dilator 140 into the access site 14 and advancing the 4F dilator 140 through the dura matter 26. The surgeon then withdraws the 4F dilator 140 from the access site 14, the sheath 120, and the first guidewire 110. The surgeon then introduces a 5F dilator 140 onto the proximal end of the first guidewire 110 and introduces the 5F dilator 140 into the lumen of the dilator 140 starting at the proximal end 146 of the dilator 140. The surgeon advances the 5F dilator 140 through the tip 124 of the sheath 120 until the tip 144 of the 5F dilator 140 is near the access site 14. The surgeon continues dilating the access site 14 by advancing the tip 144 of a 5F dilator 140 into the access site 14 and advancing the 5F dilator 140 through the dura matter 26. The surgeon then withdraws the 5F dilator 140 from the access site 14, the sheath 120, and the first guidewire 110. The same process is repeated for the 6F and 7F dilators. In alternate embodiments dilation of the access site 14 may be achieved using a single dilator, an expandable dilator, and/or a balloon catheter, for example. Dilation of the access site 14 should not exceed the outer diameter of the sheath 120. If the access site 14 is over dilated, the sheath 120 may not seal against the opening in the dura matter 26 and CSF may leak through the opening around the sheath 120, for example.

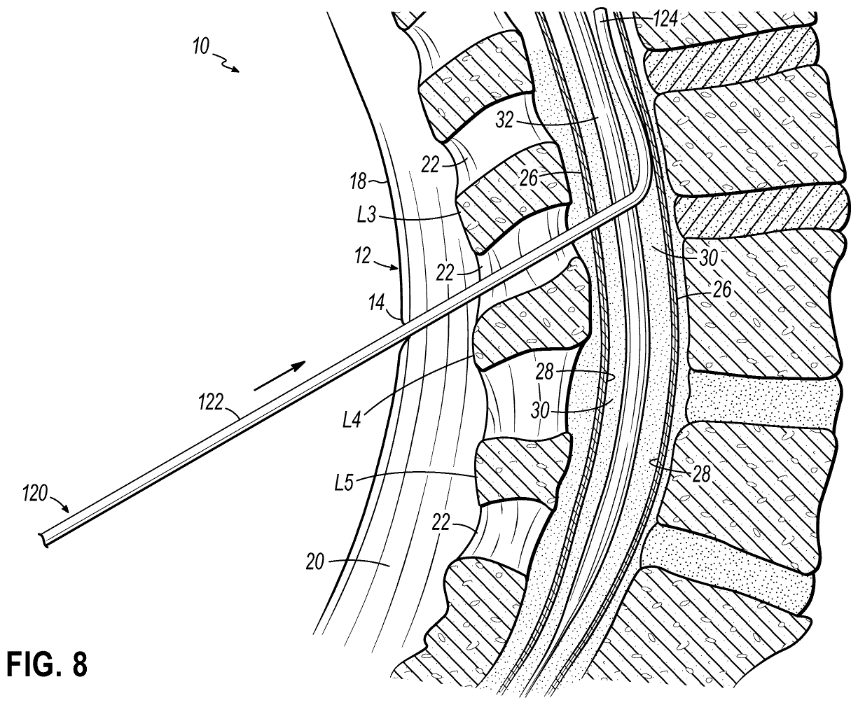

FIG. 8 is an illustrative view of the sheath 120 in the subarachnoid space 30 anterior to a spinal cord 32 of the patient 10. In this illustrative method, after the access site 14 is dilated, the tip 124 of the sheath 120 is advanced into the subarachnoid space 30 anterior to the spinal cord 32 with the tip 124 of the sheath 120 directed generally up the patient's spinal column 32 toward the brain 34. After the sheath 120 is positioned in the subarachnoid space 30 anterior to the spinal cord 32, as shown in FIG. 8, the first guidewire 110 and/or the dilator 140 are removed from the sheath 120. In this orientation, this sheath 120 is the primary access for subsequent procedures as described herein. Once the sheath 120 is positioned in the subarachnoid space 30 anterior to the spinal cord 32, the sheath 120 provides a protected path for other devices to be directed to the brain 34 without risk of damaging nerves, blood vessels, or other tissue as they are protected by the sheath 120. The surgeon may use the side port 130 of the sheath 120 to inject contrast through the sheath lumen to aid in visualization using medical imaging during the procedures described herein.

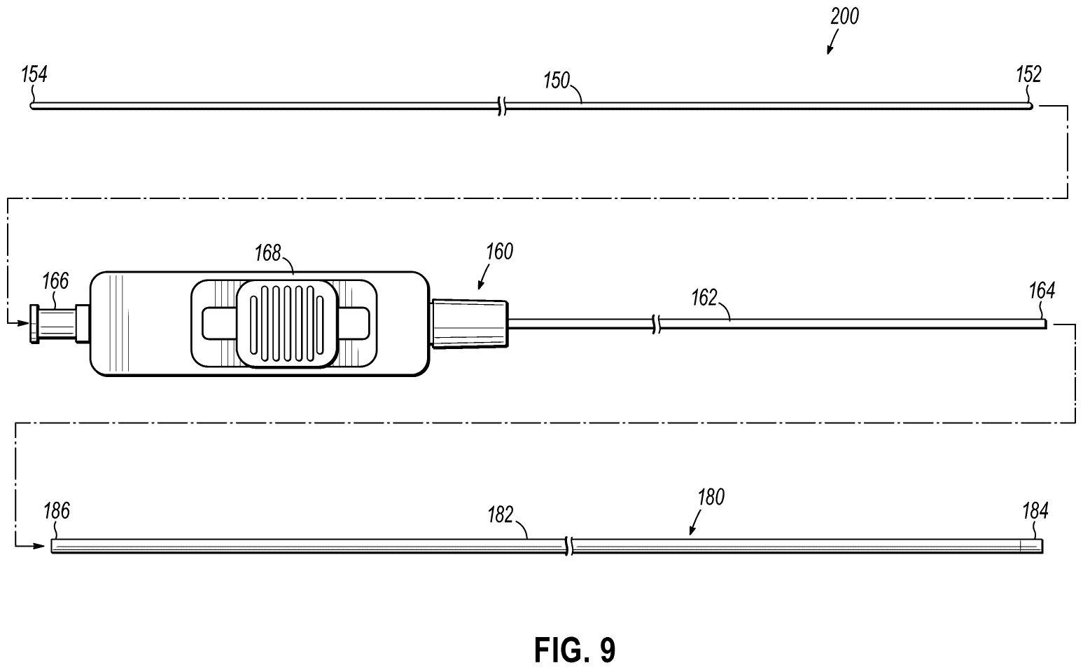

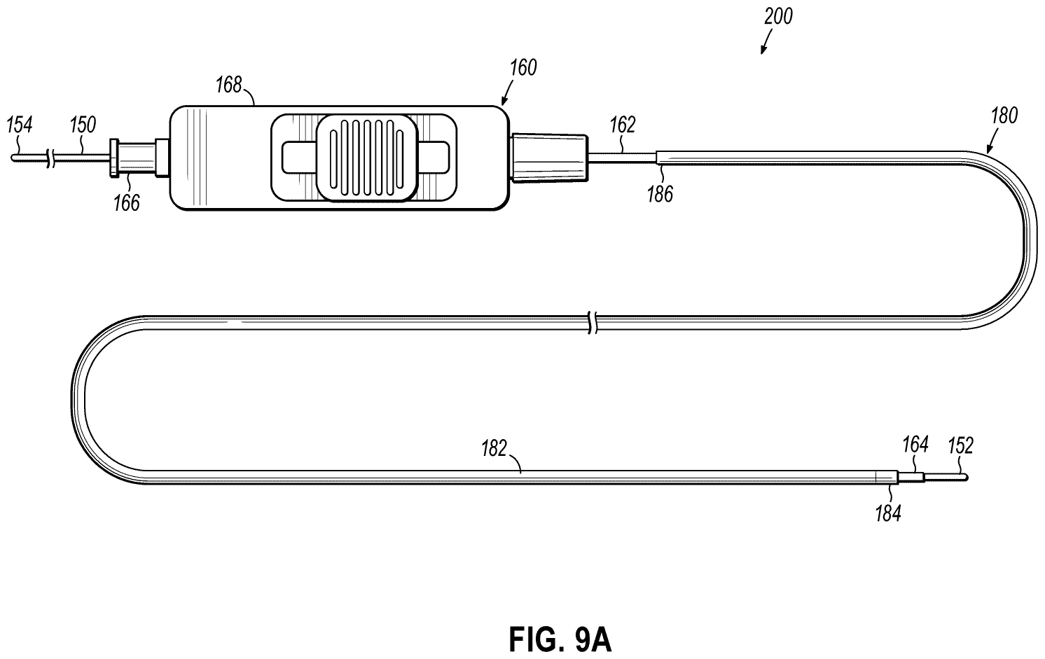

FIG. 9 is an illustrative view of a second guidewire 150 being introduced into a first catheter 160, and the first catheter 160 being introduced into a second catheter 180, and FIG. 9A is an illustrative view of an assembly of the second guidewire 150, first catheter 160, and second catheter 180, all according to at least some aspects of the present disclosure.

In this illustrative example, the second guidewire 150 includes a tip 152 located at a distal end of the second guidewire 150, and a proximal end 154, and is approximately 0.014″ diameter. The second guidewire 150 may be a stiff and/or an extra support coronary chronic total occlusion wire, such as a Stryker Neurovascular Synchro®, or an Abbott HI-TORQUE CROSS-IT™, for example. In this illustrative method, the second guidewire 150 is used to aid navigation of the catheters 150, 160 and for crossing the membrane floor of the third ventricle as described herein.

In this illustrative example, the first catheter 160 includes an elongated shaft 162, a tip 164 located at a distal end of the shaft 162, a proximal end 166, and a first catheter lumen extending longitudinally through the first catheter 160 from the proximal end 166 to the tip 164. The first catheter lumen is generally located along a longitudinal centerline of the shaft 162 and is configured to slidably receive medical devices such as guidewires, catheters, and/or dilators, for example. In this illustrative example, the first catheter 160 is approximately 150 cm long. In some embodiments, the first catheter 160 may be a steerable microcatheter, such as a Bendit® Technologies microcatheter, for example. In some exemplary embodiments, at least a portion of the first catheter 160 may be constructed of a radiopaque material which may facilitate visualization of the first catheter 160 using various medical imaging modalities.

In this illustrative example, the first catheter 160 is a steerable catheter and includes a steering mechanism 168 located at the proximal end 166 of the first catheter 160. The steering mechanism 168 is used to steer the tip 164 of the first catheter 160. In some embodiments, the steering mechanism 168 is configured, and may be used, to steer the tip 164 in a single direction. If the steering mechanism 168 is configured to steer the tip 164 in a single direction, a user may twist or rotate the first catheter 160 around the longitudinal axis of the first catheter 160, thereby rotating the tip 164, and allowing the user to use the steering mechanism 168 to steer the tip 164 in an alternate direction, for example. It is preferable that the first catheter 160 has good torque response, i.e., when the surgeon twists the proximal end of the 166 of the first catheter 160, the tip 164 of the first catheter 160 will twist an equal amount such that the tip rotation is equal to the proximal end rotation. In some embodiments, the steering mechanism 168 may be used to steer the tip 164 in a first direction and a second direction, for example. In some embodiments, the first direction and the second direction may be in a first steering plane and the first direction may be generally opposite the second direction, for example. In some embodiments, the steering mechanism 168 may be used to steer the tip 164 in a third direction and a fourth direction, for example. In some embodiments, the third direction and the fourth direction may be in a second steering plane and the third direction may be generally opposite the fourth direction, for example. In some embodiments, the first steering plane may be generally perpendicular to the second steering plane, for example.

In this illustrative example, the second catheter 180 includes an elongated shaft 182, a tip 184 located at a distal end of the shaft 182, a proximal end 186, and a second catheter lumen extending longitudinally through the second catheter 180 from the proximal end 186 to the tip 184. The second catheter lumen is generally located along a longitudinal centerline of the shaft 182 and is configured to slidably receive medical devices such as guidewires, catheters, and/or dilators, for example. In this illustrative example, the second catheter 180 is a distal access catheter and is approximately 132 cm long. In some embodiments, the second catheter 180 may be a neurovascular distal access catheter such as a Stryker Neurovascular AXS Catalyst® 5, for example. In some exemplary embodiments, at least a portion of the second catheter 180 may be constructed of a radiopaque material which may facilitate visualization of the second catheter 180 using various medical imaging modalities.

In this illustrative method, the second guidewire 150 is introduced into the lumen of the first catheter 160, and the first catheter 160 is introduced into the lumen of the second catheter 180 resulting in a coaxial catheter system 200. The inside diameter of the lumen of the second catheter 180 should only be large enough to accommodate the outer diameter of the first catheter 160, allowing just enough clearance for the first catheter 160 to slide and rotate within the lumen of the second catheter 180. It should be noted that a gap between the inside diameter of the lumen of the second catheter 180 and the outer diameter of the first catheter 160 can “grab” tissue or hang up on nerves or blood vessels during a procedure. The first catheter 160 must be longer in length than the second catheter 180 so that the tip 164 of the first catheter 160 extends past the tip 184 of the second catheter 180 and the steering capabilities of the first catheter 160 can be used to guide the second catheter 180.

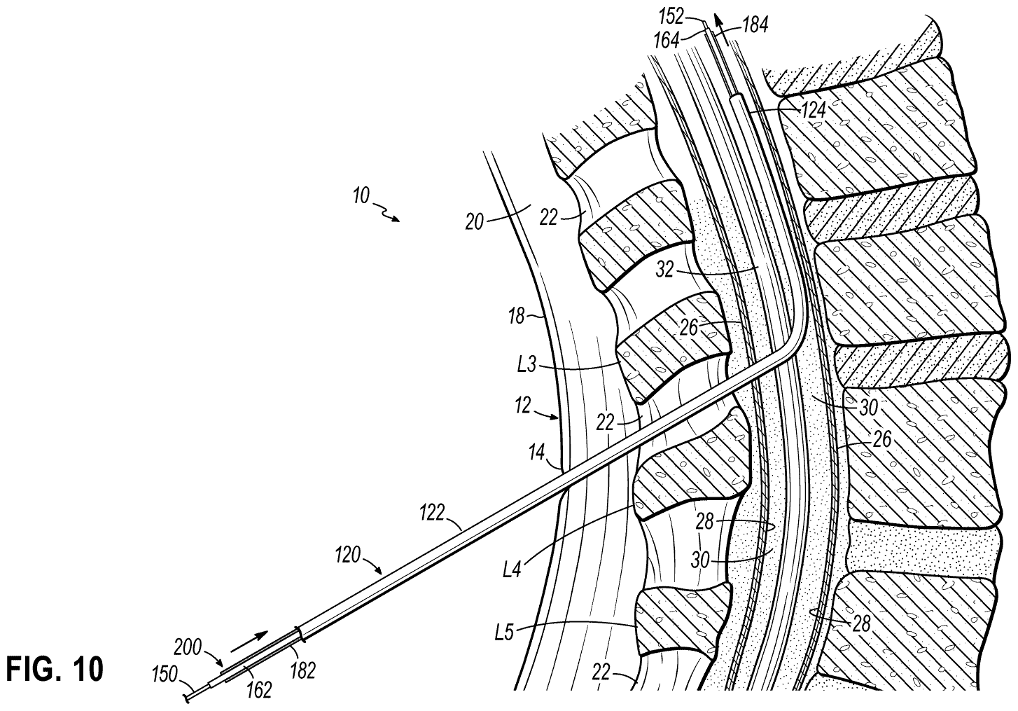

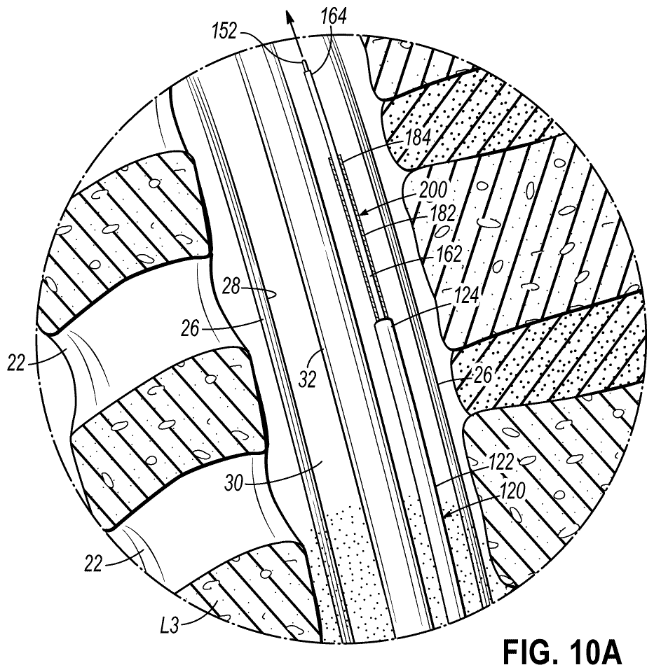

FIG. 10 is an illustrative view of the second guidewire 150, the first catheter 160, and the second catheter 180 being directed through the sheath 120, and FIG. 10A is an enlarged view of the tips of the second guidewire 150, the first catheter 160, and the second catheter 180 in the subarachnoid space 30 anterior to a spinal cord 32, according to at least some aspects of the present disclosure. In this illustrative method, the coaxial catheter system 200 including the second guidewire 150, the first catheter 160, and the second catheter 180 is directed through the sheath 120 and into the subarachnoid space 30 anterior to a spinal cord 32 of the patient 10. In alternate methods, the second guidewire 150 may be directed into the sheath 120, and the first catheter 160 and the second catheter 180 may be directed over the proximal end 154 of the second guidewire 150 to direct the first catheter 160 and the second catheter 180 through the sheath 120

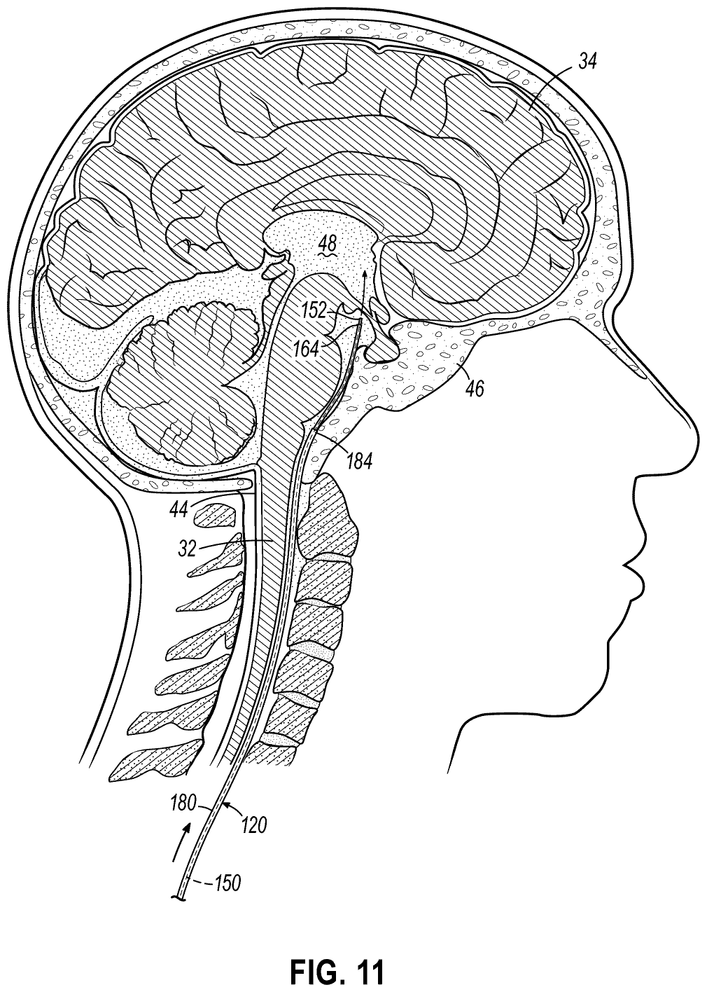

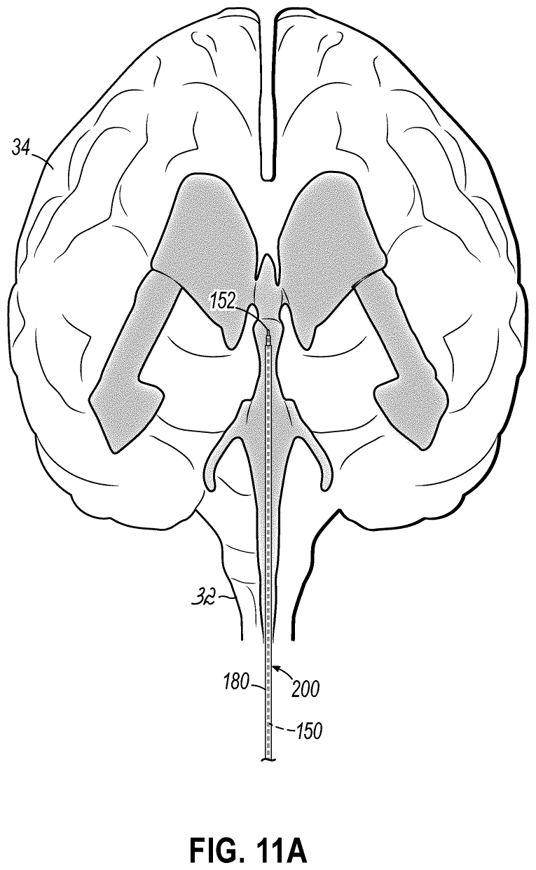

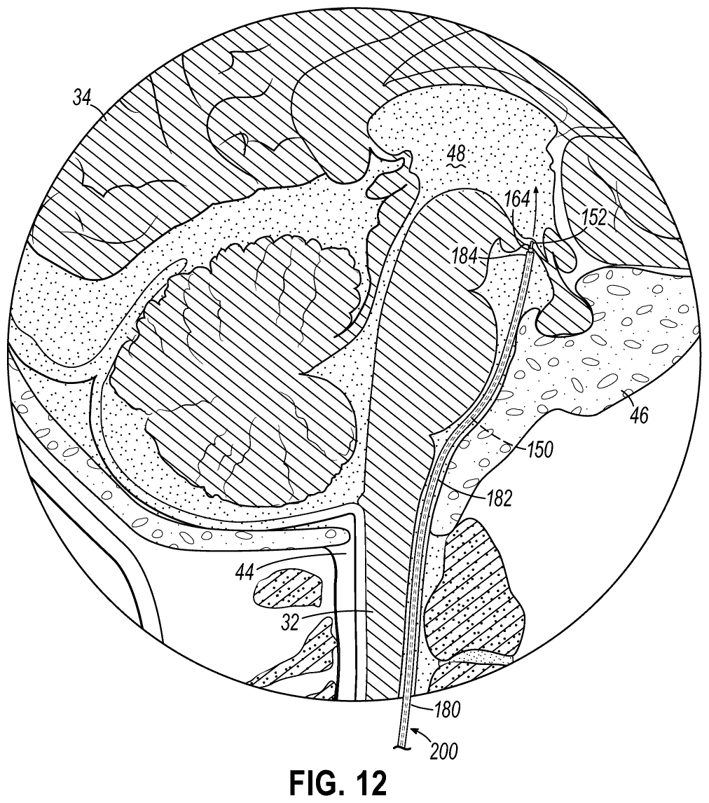

FIG. 11 is an illustrative view of the second guidewire 150, the first catheter 160, and the second catheter 180 being directed along the anterior side of the spinal cord 32, FIG. 11A is an illustrative view of the second guidewire 150, the first catheter 160, and the second catheter 180 being directed along the midline of the brain 34, and FIG. 12 is an illustrative view of the second guidewire 150, the first catheter 160, and the second catheter 180 being directed within a brain 34 of the patient 10, all according to at least some aspects of the present disclosure. In this illustrative method, the coaxial catheter system 200 is navigated into the brain 34 along the central canal 44, with tips 152, 164, 184 pointing towards the clivus 46 (bony structure) and away from the basilar artery. It is important that when advancing the coaxial catheter system 200 within the brain 34 that the coaxial catheter system 200 remains along a mid-line of the brain 34 and avoids the two lateral recesses to the left and right of the mid-line of the brain 34. In this illustrative method, the coaxial catheter system 200, along with the patient's anatomy, is visualized for the surgeon using one or more medical imaging techniques such as fluoroscopic and/or computed tomography imaging, for example, while the surgeon guides the coaxial catheter system 200. In some embodiments, the method may include co-registering with previously obtained magnetic resonance imaging (MRI) to use as a guide. Generally, the coaxial catheter system 200 is directed to the brain 34 and then when within the brain 34, as much as possible, along bony structures as opposed to along areas containing nerves and blood vessels to avoid injury to other anatomical structures such as blood vessels and/or nerve structures, for example. In some circumstances, it may be advantageous to advance the second guidewire 150 and then advance the first catheter 160 and/or the second catheter 180 along the second guidewire 150 in a stepwise manner.

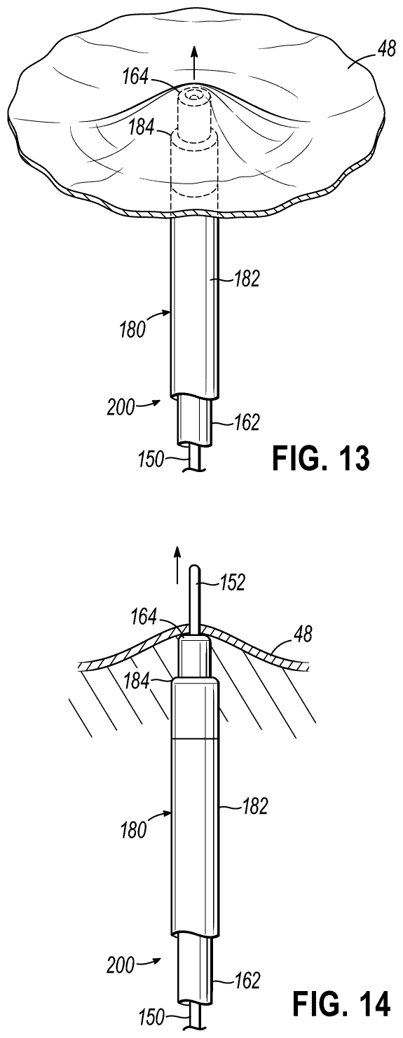

FIG. 13 is an illustrative view of the first catheter 160 “tenting” the membrane floor 48 of the third ventricle 50, and FIG. 14 is an illustrative view showing advancing the second guidewire 150 to the tip 164 of the first catheter 160 and advancing the second guidewire 150 and the first catheter 160 through the membrane floor 48 of the third ventricle 50 to create an opening in the membrane, all according to at least some aspects of the present disclosure.

In this illustrative method, as the coaxial catheter system 200 approaches the third ventricle 50 the surgeon will guide the coaxial catheter system 200 between two mamillary bodies and engage the membrane floor 48 of the third ventricle 50 with the tip 164 of the first catheter 160. Before engaging the membrane, the surgeon will withdraw the tip 152 of the second guidewire 150 from the tip 164 of the first catheter 160. In some embodiments, pulling the tip 152 of the second guidewire 150 back from the tip 164 of the first catheter 160 may improve the steering of the first catheter 160. Guiding the coaxial catheter system 200 between the two mamillary bodies may include torquing (turning/rotating) the first catheter 160 within the second catheter 180 to position the tip 164 of the first catheter 160 between the two mamillary bodies. Engaging the membrane with the tip 164 of the first catheter 160 may include torquing (turning/rotating) the first catheter 160 within the second catheter 180.

In a patient with hydrocephalus, the natural pressure of the CSF will cause the floor of the third ventricle, which is a membrane about 5 mm in width/diameter and about 20 microns thick, to bulge in the direction of the approaching first catheter tip 164. As used herein to describe various methods, “membrane” may refer to the floor of the third ventricle or other similar biological membranes. With the tip 164 of the first catheter 160 engaged with the membrane, the membrane, will “tent” and can be seen with a contrast injection using an imaging modality. In this illustrative method, the surgeon advances the tip 152 of the second guidewire 150 to the tip 164 of the first catheter 160 and uses the tip 152 of the second guidewire 150 to pierce the membrane, creating an initial opening. In some circumstances, the second guidewire 150 may be at least partially retracted while creating the initial opening. After piercing the membrane, the surgeon advances the tip 152 of the second guidewire 150 and the tip 164 of the first catheter 160 through the membrane. In some circumstances, the second guidewire 150 may be advanced through the membrane before the tip 164 of the first catheter 160. Once the tip 152 of the second guidewire 150 and the tip 164 of the first catheter 160 are positioned in the third ventricle 50, the surgeon will advance the tip 184 of the second catheter 180 through the membrane.

Alternatively, the surgeon may use an energizing element to create the initial opening in the membrane. An energizing element proximate to the tip 164 of the first catheter 160 and/or the tip 184 of the second catheter 180 may be energized (e.g., by applying RF and/or electrocautery energy) prior to and/or during advancement of the tip 164 of the first catheter 160 and/or the tip 184 of the second catheter 180 through the membrane. In some embodiments, an RF guidewire may be used to create the initial opening in the membrane, for example.

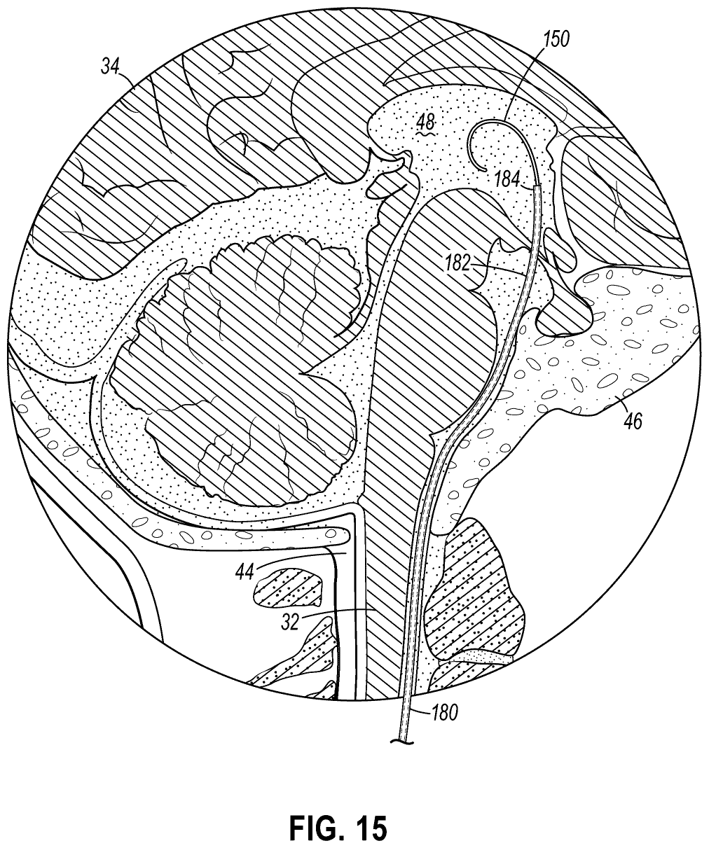

FIG. 15 is an illustrative view showing the removal of the first catheter 160 from the second catheter 180 while leaving the tip 152 of the second guidewire 150 and the tip 184 of the second catheter 180 in the third ventricle 50, according to at least some aspects of the present disclosure. In this illustrative method, the surgeon removes the first catheter 160 leaving the tip 152 of the second guidewire 150 in the third ventricle 50 or one of the lateral ventricles, and leaves the tip 184 of the second catheter 180 in the third ventricle 50. This configuration of the second catheter 180 creates a protective conduit through the lumen of the second catheter 180 along the spinal cord 32 to the brain 34 thereby minimizing potential damage to brain and spinal column structures and nerves.

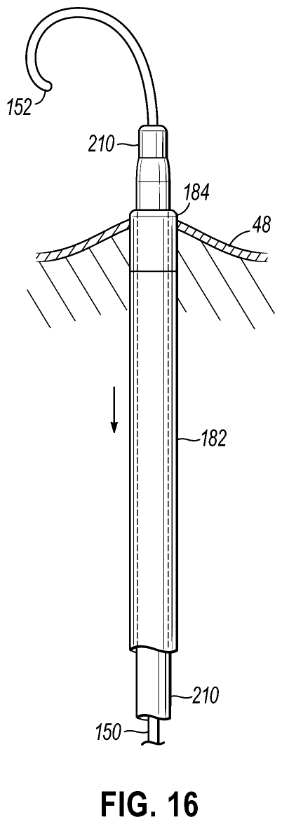

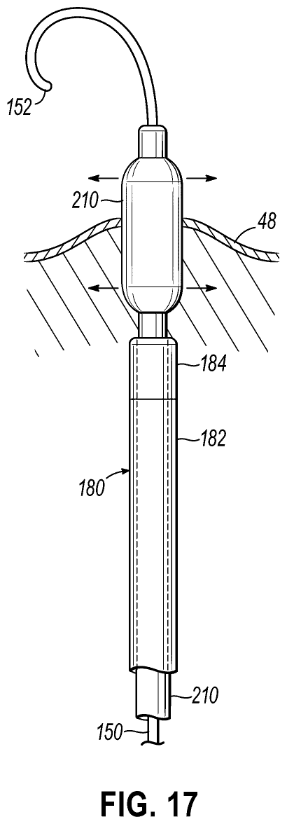

FIG. 16 is an illustrative view of the second catheter 180 being retracted and an expandable element 210 being centered in the opening in the membrane floor 48 of the third ventricle 50, and FIG. 17 is an illustrative view showing expanding expandable element 210 to expand the opening in the membrane, all according to at least some aspects of the present disclosure. In this illustrative method, the surgeon will introduce an expandable element 210 into the lumen of the second catheter 180 and advance the expandable element 210 into the third ventricle 50 and through the tip 184 of the second catheter 180. In this illustrative method, the expandable element 210 is an inflatable balloon, such as a balloon catheter, for example. In some embodiments, the expandable element 210 may be an Eclipse or Transform balloon catheter, for example. In some embodiments, the expandable element 210 should have the capability to expand to at least 5 mm in diameter. In this illustrative example, the expandable element 210 is longer that the second catheter 180. The expandable element 210 may be other devices configured to dilate openings in biological tissue. The expandable element 210 is used to dilate the opening in the membrane. The surgeon will retract the second catheter 180 through the opening in the membrane and will center the expandable element 210 in the opening in the membrane. The surgeon will expand the expandable element 210 to dilate the opening in the membrane. If the expandable element 210 is a balloon, expanding the expandable element 210 will include inflating the balloon.

After the opening in the membrane has been dilated, the expandable element 210 is retracted, and the second catheter 180 is advanced to capture the expandable element 210. If the expandable element 210 is a balloon, retracting the expandable element 210 will include deflating the balloon. In this illustrative method, the dilated opening in the membrane will allow CSF to drain from the third ventricle 50 and into the central canal 44. After the expandable element 210 has been captured, the expandable element 210 may be removed from the second catheter 180. In some embodiments, the expandable element 210 may be removed with the second guidewire 150, the second catheter 180, and/or the sheath 120 as described herein.

In some embodiments, contrast media, a dye or other substance that helps show areas, fluids, and/or tissue inside the body, is injected into the third ventricle 50 through the second catheter 180 to confirm that CSF is draining from the third ventricle 50 into the interpeduncular or basal cistern 52, which is one of the subarachnoid cisterns, and eventually into the central canal 44. Drainage of the excess CSF from the third ventricle 50 will typically be slow and may take a few days to complete depending on the patient. The contrast media may assist medical personnel in determining the progress of the CSF drainage from the third ventricle 50 into the interpeduncular cistern 52 and the central canal 44.

The next step in the illustrative procedure includes removing the second guidewire 150, the second catheter 180, and the sheath 120 from the access site 14. In some embodiments, the second guidewire 150 and the second catheter 180 may be removed simultaneously from the sheath 120, followed by the sheath 120 being removed from the access site 14. In some embodiments, the second guidewire 150 and the second catheter 180 may be removed individually from the sheath 120, followed by the sheath 120 being removed from the access site 14. In some embodiments, the second guidewire 150, the second catheter 180, and the sheath 120 may be removed simultaneously from the access site 14. Regardless of the order of removal of the second guidewire 150, the second catheter 180, and the sheath 120 from the access site 14, it is important that the spinal cord 32 and other tissues and structures are not injured or damaged during the removal of the devices.

After all devices have been removed from the access site 14, a blood patch, a surgical procedure that uses autologous blood to close a hole in the dura matter 26 to prevent leakage of CSF, is applied to close the access site opening in the dura matter 26.

In alternate methods, one or more light elements may be employed along with at least some of the devices and methods described above. Treatments for some medical conditions may include Photobiomodulation (PBM Therapy) which is the application of red and near infra-red light for medical treatment. The light can originate from a laser or a light emitting diode (LED), and can be pulsed and/or continuous. The health benefits of PBM Therapy are well documented. At least some of the devices and methods described above may be used to deliver PBM therapy directly to the tissue to be treated, and specifically the brain and/or spinal tissue.

A method to deliver PBM therapy may include advancing the tip 164 of the first catheter 160 and/or the tip 184 of the second catheter 180 to an area of the spinal column and/or brain of a patient where PBM therapy is desired, similar to the methods described above. In some methods, a surgeon may advance one or more light elements to the tip 164 of the first catheter 160 and/or the tip 184 of the second catheter 180. The one or more light elements are configured to deliver the light to biological tissues. Once in a location determined by the surgeon, the one or more light elements may be energized by the surgeon to apply light therapy to biological tissues such as brain or spinal tissue, for example. The one or more light elements may be configured to receive light via an optical fiber harness, for example. Depending on the surgical technique that is employed, the optical fiber harness may comprise one or more separate optical fibers. In some embodiments, the one or more light elements may be constructed of radiopaque materials, which may facilitate visualization of the one or more light elements using various medical imaging modalities.

The present disclosure generally discloses “over-the-wire” and steerable catheter techniques for performing a catheter based procedure. It will be understood that other catheter based techniques may be used instead for delivering instruments and components as generally described. These techniques may include, for example, “rapid exchange” techniques in which a catheter includes a short guide wire receiving sleeve or inner lumen extending through the distal portion of the catheter. The structure of the rapid exchange catheter allows for the quick exchange of the catheter without the need for the use of an exchange wire or adding a guide wire extension to the proximal end of the guide wire. This type of catheter system may be used for similar benefits in procedures as generally shown and described in this disclosure.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A method of performing a ventriculostomy, the method comprising:
creating an access site in tissue enveloping a spinal cord in a lumbar region;
inserting a guidewire into the access site;
introducing a sheath over the guidewire;
introducing a dilator over the guidewire;
dilating the access site;
inserting the sheath into the access site and positioning a tip of the sheath in a subarachnoid space of a spinal column;
removing the dilator;
introducing a first catheter into the sheath and through the access site;
introducing a second catheter into the sheath and through the access site;
guiding the first catheter and the second catheter proximate to a ventricle membrane of a ventricle;
engaging the ventricle membrane with a tip of the first catheter;
creating a hole in the ventricle membrane;
advancing at least one of the first catheter or the second catheter through the hole in the ventricle membrane;
advancing an expandable element through the hole in the ventricle membrane, centering the expandable element in the hole, expanding the expandable element to dilate the hole, and contracting the expandable element;
injecting contrast into the ventricle to confirm cerebrospinal fluid is draining into a subarachnoid cistern;
removing the expandable element;
removing the second catheter;
removing the first catheter;
removing the guidewire;
removing the sheath; and
applying a blood patch to the access site.

2. The method of claim 1, wherein the first catheter is a steerable catheter.

3. The method of claim 1, wherein the guidewire is a first guidewire and the method further comprises:
introducing a second guidewire into the sheath and through the access site;
guiding the second guidewire proximate to the ventricle membrane;
advancing the second guidewire through the hole created in the ventricle membrane;
removing the second guidewire.

4. The method of claim 1, wherein guiding the second guidewire, the first catheter, and the second catheter proximate to the ventricle membrane includes visualizing at least one of the second guidewire, the first catheter, and the second catheter using an imaging device.

5. The method of claim 4, wherein visualizing at least one of the second guidewire, the first catheter, and the second catheter includes co-registering with magnetic resonance imaging.

6. The method of claim 1, wherein guiding the second guidewire, the first catheter, and the second catheter proximate to the ventricle membrane includes visualizing the ventricle membrane using an imaging modality.

7. The method of claim 6, wherein the imaging modality includes computed tomography.

8. The method of claim 6, wherein the imaging modality includes fluoroscopic computed tomography.

9. A system for performing a ventriculostomy, the system comprising:
a needle for performing a lumbar stick to create an access site in tissue enveloping a spinal cord in a lumbar region;
a guidewire to be inserted through the access site;
a sheath to be inserted through the access site over the guidewire and into a subarachnoid space of a spinal column anterior to a spinal cord;
a dilator for use with the sheath to dilate the access site allowing the sheath to be inserted through the access site;
a first catheter;
a second catheter to receive the first catheter, and the first catheter and the second catheter are inserted through the sheath into the subarachnoid space of the spinal column and guided proximate to a ventricle membrane; and
an expandable element to dilate a hole in the ventricle membrane.

10. The system of claim 9, wherein the first catheter is a steerable catheter.

11. The system of claim 9, wherein the guidewire is a first guidewire;
the system further comprising
a second guidewire to be guided proximate to the ventricle membrane and inserted through the hole in the ventricle membrane.

12. The system of claim 9, wherein the needle is an 18 to 21 gauge needle.

13. The system of claim 9, wherein the first guidewire has a diameter, and the first guidewire diameter is 0.014″ to 0.025″.

14. The system of claim 9, wherein the first guidewire has a length and the first guidewire length is 70 to 100 cm.

15. The system of claim 9, wherein the dilator is a 4F to 7F dilator.

16. The system of claim 9, wherein the sheath has a length, and the sheath length is 40 to 60 cm.

17. The system of claim 9, wherein the second guidewire has a diameter, and the second guidewire diameter is 0.014″.

18. The system of claim 9, wherein the second catheter is a neurovascular distal access catheter.

19. A method of applying light therapy to at least one of an area of a brain or an area of a spinal cord, the method comprising;
creating an access site in tissue enveloping a spinal cord in a lumbar region;
inserting a guidewire into the access site;
introducing a sheath over the guidewire;
introducing a dilator over the guidewire;
dilating the access site;
inserting the sheath into the access site and positioning a tip of the sheath in a subarachnoid space of a spinal column;
removing the dilator;
introducing a first catheter into the sheath and through the access site;
introducing a second catheter into the sheath and through the access site;
advancing at least one of the first catheter or the second catheter proximate to one of the area of the brain or the area of the spinal cord;
energizing a light element;
applying light to the area of the brain or the area of spinal cord tissue
removing the second catheter;
removing the first catheter;
removing the guidewire;
removing the sheath; and
applying a blood patch to the access site.

20. A system for applying light therapy to at least one of an area of a brain or an area of a spinal cord, the system comprising:
a needle for performing a lumbar stick to create an access site in tissue enveloping a spinal cord in a lumbar region;
a guidewire to be inserted through the access site;
a sheath to be inserted through the access site over the guidewire and into a subarachnoid space of a spinal column anterior to a spinal cord;
a dilator for use with the sheath to dilate the access site allowing the sheath to be inserted through the access site;
a first catheter;
a second catheter to receive the first catheter, and the first catheter and the second catheter are inserted through the sheath into the subarachnoid space of the spinal column and guided proximate to a ventricle membrane; and
a light element for applying light therapy to the area of the brain or the area of the spinal cord.
